(19) United States
(12) Patent Application Publication
(10) Pub. No.: US 2010/0324481 A1
(43) Pub. Date: Dec. 23, 2010

(54) ULTRASOUND PUMPING APPARATUS FOR USE WITH THE HUMAN BODY

(75) Inventor: Eilaz Babaev, Minnetonka, MN (US)

Correspondence Address: Bacoustics, LLC, 5929 BAKER ROAD, SUITE 470, MINNETONKA, MN, 55345, US

(73) Assignee: BACOUSTICS, LLC, Minnetonka, MN (US)

(21) Appl. No.: 12/861,362

(22) Filed: Aug. 23, 2010

Related U.S. Application Data
(63) Continuation-in-part of application No. 11/777,955, filed on Jul. 13, 2007, now Pat. No. 7,780,095.

(51) Int. Cl. A61M 31/00

(52) U.S. Cl. 604/93.01

(57) ABSTRACT

An apparatus utilizing ultrasonic vibrations to force the movement of protrusions to supply a liquid stream with sufficient velocity as to penetrate deep into treatment areas including body lumens, body cavities, and poorly perfused tissues at a pressure not harmful to the treatment area into which the liquid stream is injected is presented. The apparatus comprises an ultrasound horn with a dampening grommet attached to its distal end and an internal chamber. Within the internal chamber of the horn are protrusions extending into the chamber. Liquid to be expelled from the horn enters the internal chamber of the horn through at least one channel passing through a wall of the horn and leading into the chamber. After passing through the horn's internal chamber the liquid exits the horn by passing through a channel originating in the front wall of the chamber.

### CROSS REFERENCE TO RELATED APPLICATIONS

This application is a continuation-in-part of pending U.S. application Ser. No. 11/777,955 filed Jul. 13, 2007, the teachings of which are hereby incorporated by reference.

### BACKGROUND OF THE INVENTION

1. Field of the Invention

The present invention relates to an ultrasound liquid delivery device useful for introducing liquids into lumens and cavities of the body.

2. Description of the Related Art

Various maladies such as infections, conditions, ailments, and diseases of the body are difficult to treat pharmacologically due to inaccessibility of the site of the malady to pharmacologically active compounds or drugs such as, but not limited to, chemical compounds, chemicals, small molecules, proteins, and genes. Such sites include body lumens and body cavities such as, but not limited to, the urethra, sinuses, vagina, uterus, outer ear, lungs, thoracic cavity, and colon, and poorly vasculated tissue such as, but not limited, to bones. Though commonly considered otherwise by those outside of the biological arts, lumens and body cavities are outside of the body. This somewhat paradoxical statement can best be understood by considering the path an orally taken drug travels to reach the urethra, a component of the urinary tract.

A pill containing the drug is first dissolved in the stomach or small intestine after being swallowed, thereby liberating small drug molecules. The small drug molecules are then carried into the cells forming the walls of the small intestine by transport molecules on the cells' surfaces. Alternatively the drug may pass directly into the cells of the small intestine on their own by either diffusing through cellular membranes or passing through pores located within the cellular membranes. Once within the cells forming the walls of the small intestine, the drug molecules are transported by similar mechanisms into the patient's blood stream. The drug molecules then spread throughout the patient's entire blood stream where they are subjected to degradation and inactivation by various elements within the blood. The blood stream also transports the drug molecules to potential sites of degradation, inactivation, and excretion such as the kidneys and liver. Despite dilution by wide spread distribution, inactivation, degradation, and excretion, some of the drug molecules reach areas of the blood stream adjacent to the urethra. However, to effectively treat a malady within the urethra, the drug must by taken out the bloodstream by the cells forming the urethra and then deposited into the open space of the urethra.

With respect to maladies within poorly vasculated or perfused tissue, pharmacological treatment is limited by a poor blood supply. Most methods of drug delivery rely upon the blood stream to deliver the drug molecules to the site of the malady to be treated. Consequently the amount of drug delivered is positively related to the amount of blood delivered to the site of the malady. Therefore, a location or tissue of the body that receives a diminished, reduced, compromised, or small supply of blood is less accessible to pharmacological treatment. Various medical devices and procedures have been developed to supplement pharmacological treatment of maladies located within lumens, open body cavities, and poorly perfused tissues.

Catheter drug delivery methods and devices have been developed to overcome the limitations of pharmacological treatment. Inserting catheters into lumens or body cavities and injecting a drug through the catheters gives pharmacologically active compounds access to the site of a malady. Running a catheter through a small or narrow body lumen, such as the urethra, can be uncomfortable for the patient. Inflicting injury while inserting the catheter can create serious complications such as allowing an infection on the inner wall of the lumen or body cavity to enter the blood stream, thereby spreading the infection throughout the body. Furthermore, finding the site of the malady to be treated, while inserting the catheter, can be difficult.

To get pass the delivery limitations of drugs for the treatment of maladies within shallow lumens or cavities of the body creams and other medical ointments have been developed. The cream or ointment is spread on the walls of the cavity with a finger, swab, or similar device. Though these creams and ointments can be effective, their use is generally not preferred by the patient as they can be messy, leak out, and are often unpleasant to apply.

Delivering drugs to open body cavities and lumens not sufficiently accessible as to permit the application of creams, such as the sinuses, has been accomplished with various spray and misting devices. Exemplar devices well known in the art are nasal sprays and inhalers. Generating a spray or mist through the use of pumps or aerosols, it is difficult to utilize nasal sprays and inhalers to deliver drugs deep within an open cavity. The difficulty in achieving deep drug delivery is due to the low pressure, low velocity sprays generated by these devices. Generation of the spray or mist is generally accomplished by pressurizing the drug to be delivered and then forcing the drug through a small orifice. As to avoid damaging tissue within the cavity or lumen into which the drug is to be delivered, low pressure is used to drive drug delivery. Because pressure is the only force driving drug delivery, the use of low pressure creates a low velocity spray limiting the depth of drug penetration. Consequently, it is difficult to deliver drugs from such devices to sites of maladies deep within body cavities or lumens.

U.S. Pat. No. 6,601,581 to the present inventor describes an ultrasound inhaler utilizing ultrasonic energy to create a fine drug mist. Though effective at delivering drugs to the lungs and bronchia, this device is incapable of creating a spray of sufficient velocity to enable drug delivery deep within lumens and cavities of the body.

Though drugs are often effective in treating various maladies of the body, they are not always necessary. Many maladies of the body can be treated simply by washing or flushing the affected area. Washing or flushing with simple, drug free fluids such as saline or water has the benefit of avoiding the various side effects and complications drugs may possess. Unfortunately, the practicality of washing or flushing body cavities, body lumens, and poorly perfused tissues is hindered, as is drug delivery, by the lack of devices capable of delivering a liquid deep into the affected lumens, cavities, and poorly perfused tissues.

### SUMMARY OF THE INVENTION

The present invention relates to an apparatus utilizing ultrasonic vibrations to force the movement of protrusions to supply a liquid stream with sufficient velocity as to penetrate deep into treatment areas including body lumens, body cavities, and poorly perfused tissues at a pressure not harmful to the treatment area into which the liquid stream is injected. The apparatus comprises an ultrasound horn with a dampening grommet attached to its distal end and an internal chamber. Within the internal chamber of the horn are protrusions extending into the chamber. Liquid to be expelled from the horn enters the internal chamber of the horn through at least one channel passing through a wall of the horn and leading into the chamber. After passing through the horn's internal chamber the liquid exits the horn by passing through a channel originating in the front wall of the chamber. The exiting liquid stream permits the delivery of therapeutically effective drug concentrations to body lumens, body cavities, and poorly perfused tissues. Alternatively, the liquid stream may be used to wash or flush body lumens, body cavities, and poorly perfused tissues.

The present invention generates the liquid stream by coupling ultrasonic vibrations to a series of pumping members or protrusions. Ultrasonic vibrations can be provided by attaching the proximal end of the horn to an ultrasonic transducer. As the ultrasonic vibrations generated by the transducer travel through the horn segments of the horn expand and contract. Expansion and contractions is limited to segments of the horn corresponding with regions between nodes (points of minimum deflection or amplitude) on the ultrasonic vibrations. Consequently, segments of the horn corresponding directly with anti-nodes on the ultrasonic vibrations exhibit the greatest amount of movement, as anti-nodes are points of maximum deflection or amplitude. Conversely, segments of the horn corresponding exactly with nodes on ultrasonic vibrations do not expand or contract.

As segments of the horn are expanding and contracting, the protrusions which extend from those segments on the chamber's walls, also contract and expand. This causes a pumping motion as the front-facing edges of the protrusions move forward; increasing the fluid pressure and driving the liquid forward.

The rear-facing edges of the protrusions should be more streamlined than their front-facing edges to efficiently and effectively push liquid forward through the chamber. This configuration enables the net movement of the liquid (fluid pushing forward minus fluid pushing backwards) in the forward direction.

It is preferred to orient the front-facing edges of the protrusions approximately perpendicular to the central axis of the horn. A front-facing edge that is approximately perpendicular to the central axis acts more like a wall pushing liquid forward when the protrusion expands.

It is also preferred to locate at least a portion of the front-facing edges of the protrusions on anti-nodes of the ultrasonic vibrations passing through the horn. So locating the front-facing edges enables the pumping action produced by vibrating the horn to be better controlled by the frequency of the vibrations. For example, if the frequency of the ultrasonic vibrations were cut in half, then some of the front-facing edges would fall on nodes (points of no movement) of the ultrasonic vibrations. This would prevent those protrusions from pumping liquid and, overall, reduce the pumping action of the horn. Therefore, the pumping mechanism may be controlled by adjusting the frequency of the ultrasonic vibrations passing through the horn.

An important aspect of the spraying apparatus involves the relationship between the amplitude of the ultrasonic vibrations passing through the horn and the pumping behavior of the protrusions. Increasing the amplitude of the ultrasonic vibrations passing through the horn increases the degree of deflection the ultrasonic vibrations create. Therefore, the higher the amplitude of the ultrasonic vibrations passing through the horn the farther forward the protrusions will move. Consequently, increasing the amplitude will increase the amount of pumping produced by the protrusions. Increased pumping by the protrusions increases the pressure generated by the protrusions' motion. If the horn is vibrated in resonance by a piezoelectric transducer driven by an electrical signal supplied by a generator, then the amplitude of the vibrations passing through the horn can be increased by increasing the voltage of the electrical signal driving the transducer.

The protrusions may be discrete elements such as, but not limited to, discrete bands encircling the internal chamber of the ultrasound tip. The protrusions may also spiral down the chamber similar to the threading in a nut. However, the protrusions need not encircle the entire circumference of the chamber.

Protrusions may take the form of various shapes such as, but not limited to, convex, spherical, triangular, polygonal, teeth-like, or any combination thereof so long as enough of the protrusions contain a front-facing edge less streamlined than their corresponding rear-facing edge, as to generate a net forward movement of liquid passing through the internal chamber of the horn. Depending upon the chosen conformation of the protrusions, the front-facing edges of the protrusions need not be orientated approximately perpendicular to the central axis of the horn. Likewise, depending upon the conformation chosen, it may be possible to orient the rear-facing edges of the protrusions approximately perpendicular to the central axis of the horn.

It is preferable to position the back and front walls of the chamber on nodes of the ultrasonic vibrations. Positioning the back and front walls on nodes minimizes the amount of ultrasonic vibrations emanating into the chamber from the back wall and the amount of ultrasonic vibrations reflecting back into the chamber off the front wall. This is significant because the ultrasonic vibrations reflecting off the front wall pushes liquid back into the chamber. However, this is only a suggested preference since the walls of the chamber may be positioned on any point along the ultrasound vibrations.

The front wall of the chamber may contain slanted portions. A front wall with slanted portions serves to funnel liquid to be injected into the treatment area into the channel leading out of the internal chamber.

Treating maladies of lumens, cavities, and/or tissues of the body with the present invention entails first selecting an appropriate liquid. Selecting a liquid comprising a pharmaceutically active compound or drug with properties known or believed to treat the malady present may be advantageous. Alternatively, the liquid chosen may possess properties ideal for washing the treatment area of contaminants, infectious microbes, bacteria, funguses, accretions, impacted matter, dirt, debris, necrotic tissue, or other undesirable elements. Choosing a liquid capable of coupling the inner chamber of the horn with the treatment area as to allow for the transmission of ultrasonic energy from the inner chamber to the treatment area may also prove advantageous. The coupled ultrasonic energy may induce cavitations within the treatment area and help to dissolve accretions, remove undesirable elements from the treatment area, promote the growth of healthy tissue within the treatment area, retard the growth of or kill unwanted tissue within the treatment area, retard the growth of and/or kill infectious microbes within the treatment area, enhance the entry of drugs into cells within and in the vicinity of the treatment area, or provide other positive healing benefits. The liquid chosen may possess all or some of the above mentioned properties.

The ultrasound spraying apparatus of the present invention may also be used for preventative purposes. For instance, the present invention may be used to wash the treatment area as to prevent or lessen the likelihood of developing a malady within the treatment area. Delivering liquids comprising vitamins, minerals, drugs, or other elements known or believed to have a positive effect on the treatment area may also help to preserve proper functioning of the treatment area and prevent the development of maladies therein. Preventing biological processes from occurring within the treatment area, such as, but not limited to, ovulation, fertilization, or implantation, may also be accomplished by using the present invention to deliver to the treatment area liquids possessing properties known or believed to retard the undesired processes. Conversely, inducing the occurrence of wanted biological processes, such as, but not limited to, bowel movement, immune suppression, histamine inhibition, or bronchial dilation, may be accomplished by using the present invention to deliver to the treatment area liquids possessing properties known or believed to promote the desired processes.

Once a liquid has been chosen, it is loaded into a liquid supply connected to a channel passing through a wall of the horn and leading into the chamber. The liquid supply utilized may be a syringe, a pump, a reservoir with a gravity feed, a pipette, or similar devices capable of dispensing a fluid into the channel. Utilizing a liquid supply capable of delivering a set or predetermined amount of liquid or otherwise capable of indicating or allocating the amount of liquid delivered allows the user of the present to dose the amount of liquid delivered. The liquid supply may be connected to the channel leading into the internal chamber by a flexible hose. Dampening, preventing, or lessening the transmission of vibrations from horn to the liquid supply, a flexible hose may help to prevent needles or delicate tips of the liquid supply from breaking or shearing during operation.

Prior to liquid delivery, the treatment area must be made accessible if it is not already so. An accessible treatment area is one comprising a naturally occurring or created external orifice or externally accessible orifice leading into it. If the orifice leading into the treatment area comprises a sore or wound created as a result of the malady to be treated, it is considered a naturally occurring orifice. If the treatment area is inaccessible, an orifice should be created. The creation of secondary orifices allows for the delivery of the chosen liquid from multiple sites into the treatment area. Delivering liquid from multiple orifices may provide a more uniform delivery of the chosen liquid into the treatment area. Alternatively, secondary orifices may provide a point of egress for the delivered liquid enabling the drainage of the delivered liquid or fluids within the treatment area. The resulting drainage may provide for the evacuation of undesirable elements from the treatment area. The orifice leading into the treatment area, whether naturally occurring or created, may be reinforced by the implantation of cannula into the orifice, as to prevent closure of the orifice.

Having chosen an appropriate liquid, prepared the liquid supply, and, if necessary, the orifice leading into the treatment area, the user of the present invention then chooses an appropriate dampening grommet. Selecting an appropriate grommet requires consideration of the orifice extending into the treatment area. The distal end of the grommet chosen may posses an outer perimeter sufficiently small as to allow at least a portion of the grommet to be comfortably inserted into the orifice. Alternatively, the outer perimeter of chosen grommet's distal end could be sufficiently large as to allow the grommet to encompass the orifice when pressed against the patient's body. Incorporation of a second channel within the grommet allows for the drainage of the delivered liquid or fluids from the treatment area; enabling the evacuation of liberated undesirable elements from the treatment area. The selection of the grommet may also occur before or simultaneously with the selection of the appropriate liquid. At least a portion of the grommet must be capable of blocking the delivery of ultrasonic vibrations by absorbing, dampening, lessening, or preventing the transmission of vibrations from the horn distal end of the horn to patient or orifice.

After having chosen and attached the dampening grommet, a seal is formed between the grommet and an orifice leading into the treatment area. The chosen liquid is then permitted to flow into the internal chamber through the channel passing through a wall of the horn and leading into the chamber. Ultrasonic vibrations are then induced within the horn. As the liquid passes through the channel, the liquid becomes accelerated and is ejected from the distal end of the grommet into the treatment area as a higher velocity liquid stream.

### BRIEF DESCRIPTION OF THE DRAWINGS

FIG. 1 illustrates an embodiment of the ultrasonic spraying apparatus of the present invention.

FIG. 2 illustrates embodiments of the dampening grommet providing a few of the possible means of attaching the dampening grommet to the distal end of the ultrasound horn.

FIG. 3 depicts exemplar configurations of dampening grommets for use with the ultrasonic liquid delivery device of the present invention.

FIG. 4 illustrates a cross-sectional view of an alternative embodiment of an ultrasound horn for uses with the present invention.

FIG. 5 contains a flow chart depicting a method of treating maladies within lumens, cavities or tissues of the body with the ultrasonic liquid delivery device of the present invention.

FIG. 6 is an illustration of the treatment of bone marrow with the liquid delivery device of the present invention.

### DETAILED DESCRIPTION OF THE INVENTION

Preferred embodiments of the ultrasonic spraying apparatus are illustrated throughout the figures and described in detail below. Those skilled in the art will understand the advantages provided by the ultrasonic spraying apparatus upon review.

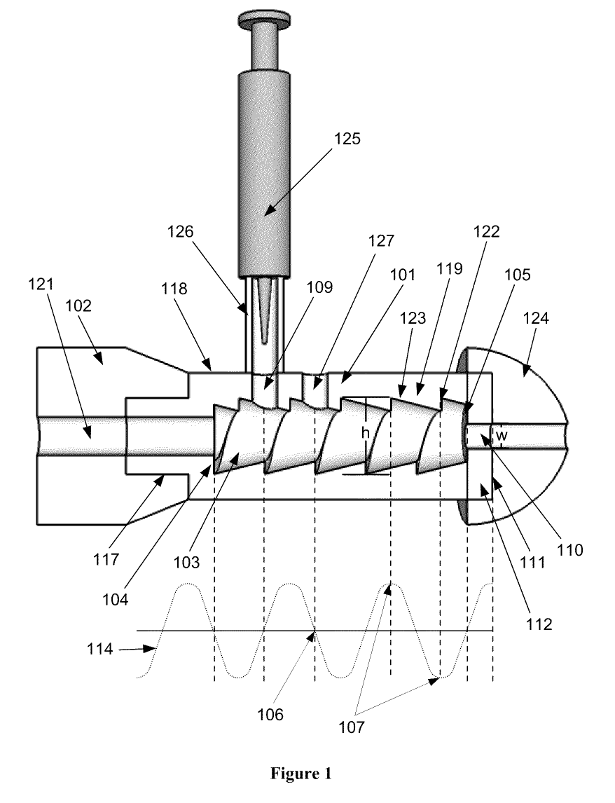

FIG. 1 illustrates an embodiment of the ultrasonic spraying apparatus comprising a horn 101 and a dampening grommet 124 attached to the distal end 111 of horn 101. Mechanically coupled to the proximal end 117 of horn 101 as to permit the induction of vibrations within horn 101 is an ultrasound transducer 102, powered by generator (not shown). As ultrasound transducers and generators are well known in the art they need not and will not, for the sake of brevity, be described in detail herein. Horn 101 may be mechanically coupled to transducer 102 by using a threaded mechanical connector, an adhesive attachment or by welding transducer 102 to horn 101. Transducer 102 and horn 101 may also be a single piece. Other manners of securing horn 101 to transducer 102, as to mechanically couple the two elements, may be equally effective and are readily recognizable to persons of ordinary skill in the art.

Ultrasound horn 101 comprises a proximal end 117, a distal end 111 opposite proximal end 117, and at least one radial surface 118 extending between proximal end 117 and distal end 111. Within horn 101 is an internal chamber 103 containing a back wall 104, a front wall 105, and a side wall extending between back wall 104 and front wall 105. The back wall 104 and front wall 105 of internal chamber 103 lie approximately on nodes 106 of ultrasonic vibrations 114. This positioning of back wall 104 and front wall 105 reduces the amount of ultrasonic vibrations within chamber 103. As nodes 106 are points on ultrasonic vibrations 114 of minimum deflection or amplitude, so positioning back wall 104 reduces its movement and collisions with the liquid within chamber 103. Similarly, positioning front wall 105 on a node reduces the echoing of ultrasonic vibrations off front wall 105. Although the preferred positions of front wall 105 and back wall 104 are approximately on nodes 106 of ultrasonic vibrations 114, front wall 105 or back wall 104 may be positioned at any point along ultrasonic vibrations 114, including anti-nodes 107.

Protrusions 119 extend from back wall 104 and spiral along side wall similar to the threading in a nut. The protrusions, however, need not encircle the entire circumference of the chamber. Protrusions 119 comprise front-facing edges 122 and rear-facing edges 123 more streamlined than their front-facing edges. Front-facing edges 122 of protrusions 119 are approximately perpendicular to the central axis of horn 101 and have portions lying approximately on anti-nodes 107 of ultrasonic vibrations 114. Although it is preferable that at least one point on front-facing edges 122 lie approximately on an anti-node, the front-facing edges may be positioned at any point along ultrasonic vibrations 114. Furthermore, not all of the front-facing edges 122 need be located on corresponding points of ultrasonic vibrations 114.

The horn may be capable of vibrating in resonance at a frequency of approximately 16 kHz or greater. The ultrasonic vibrations traveling down the horn may have an amplitude of approximately 1 micron or greater. It is preferred that the horn be capable of vibrating in resonance at a frequency between approximately 20 kHz and approximately 200 kHz. It is recommended that the horn be capable of vibrating in resonance at a frequency of approximately 30 kHz.

The signal provided by a generator driving the ultrasound transducer may be a sinusoidal wave, square wave, triangular wave, trapezoidal wave, or any combination thereof.

The liquid to be expelled from horn 101 and injected into the treatment may enter internal chamber 103 through channel 109 originating in radial surface 118 and opening into chamber 103. Channel 109 receives the liquid to be expelled from horn 101 and delivered into the treatment area from a liquid supply 125. As to facilitate entry of the liquid into chamber 103 channel 109 may lie approximately on a node 106 of ultrasonic vibrations 114.

The liquid supply 125 depicted in FIG. 1 is a typical syringe. Other liquid supplies capable of dispensing a liquid into channel 109 may be similarly effective including, but not limited to, a pump, a reservoir with a gravity feed, or a pipette. A flexible hose 126 coupling liquid supply 125 to channel 109 may be utilized to help prevent breaking or shearing of delicate tips or needles of the liquid supply by the vibrations of ultrasonic horn 101. After entering chamber 103 through channel 109, the fluid exits chamber 103 through channel 110, originating in the front wall 105 of chamber 103 and ending in the distal end 111.

If the liquid passing through horn 101 is to be atomized for inhalation or other delivery purposes, then the maximum height (h) of chamber 103 should be larger than maximum width (w) of channel 110. Preferably, the maximum height of chamber 103 should be approximately 200 times larger than the maximum width of channel 110 or greater.

As depicted in FIG. 1, horn 101 may posses multiple channels opening into the chamber that permit a fluid to be delivered into chamber 103. The additional channels may, as indicated by channel 121, open into chamber 103 at back wall 104. An additional channel, as indicated by channel 127, also open into chamber 103 at the side wall. When multiple channels with openings within a side wall of the chamber are present the openings may be aligned along the central axis of horn 101, as depicted in FIG. 1. Alternatively, the openings may be located on the same platan. Additional liquid supplies may be connected to channels 121 and 127. Alternative embodiments of an ultrasound horn 101 in accordance with the present invention may possess a single channel opening into chamber 103. When horn 101 includes multiple channels opening into chamber 103, delivery via inhalation may be improved by delivering a gas into chamber 103 through at least one of the channels.

Ultrasound horn 101 may further comprise cap 112 containing its distal end 111. Cap 112 may be mechanically attached (for example, secured with a threaded connector), adhesively attached, or welded to the distal end of horn 101. Other means of attaching cap 112 to horn 101, readily recognizable to persons of ordinary skill in the art, may be used in combination with or in the alternative to the previously enumerated means. Comprising front wall 105, channel 110, and distal end 111, a removable cap 112 permits the level of atomization to be adjusted depending on need or circumstances. For instance, the width of channel 110 may need to be adjusted to produce the desired level of atomization with different liquids. Attaching cap 112 to horn 101 on a node 106 of ultrasonic vibrations 114 passing through horn 101 may help prevent the separation of cap 112 from horn 101 during operation.

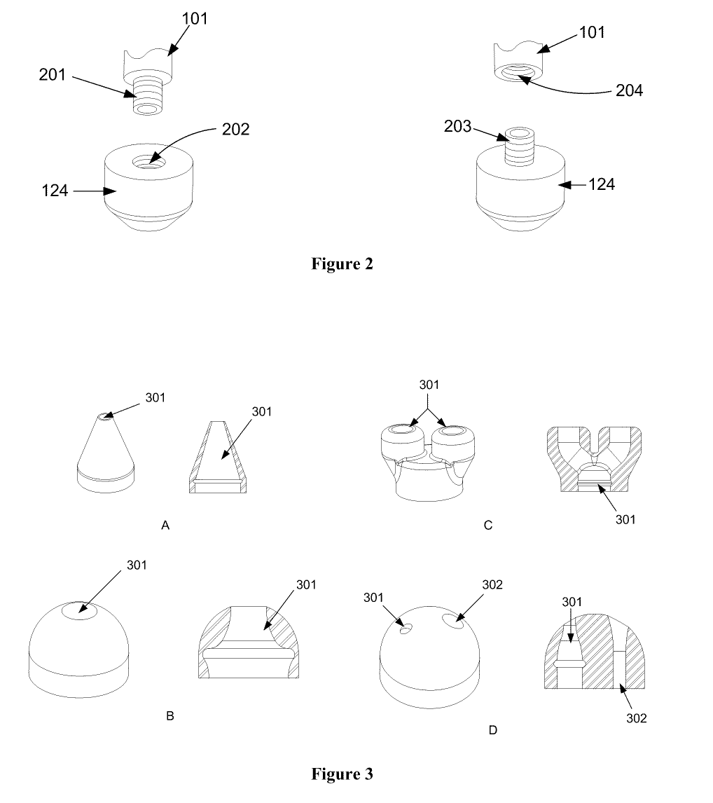

Preferably, dampening grommet 124 should be attached to horn 101 as to occlude substantially all the surface of distal end 111 as to block the delivery of the majority of vibrations emanating from the distal end to the patient or orifice. Various mechanical means may be utilized to attached grommet 124 to horn 101. As shown in FIG. 2, a threaded protrusion 201 at the distal end of horn 101 may be received by threaded recess 202 at the proximal end of grommet 124. Alternatively, a threaded protrusion 203 at the proximal end of grommet 124 may be received by a threaded recess 204 at the distal end of horn 101. It is also possible, as shown in FIG. 1, to mechanically attach grommet 124 to horn 101 by providing grommet 124 with a recess that receives the distal end 111. Alternatively, dampening grommet 124 may be attached to ultrasound horn 101 by adhesives. Other means of attaching grommet 124 to horn 101 may be utilized, provided the means chosen prevents the separation of grommet 124 from horn 101 during operation.

The distal end of the dampening grommet should be sized to fit within and form a relative seal against the orifice leading into the treatment area. Alternatively, the distal end of the dampening may be sized as to encompass the orifice leading into the treatment area and form a seal against the patient's body. Furthermore, the dampening grommet should be constructed or configured as to possess dampening properties enabling the blocking of the delivery of ultrasonic vibrations from the distal end of the horn. Providing the grommet with dampening properties may be achieved by constructing at least a portion the dampening grommet from rubber, plastic, silicon, or other compounds capable of absorbing, dampening, lessening, or preventing the transmission of vibrations from the horn distal end of the horn to patient or orifice. Alternatively, the dampening grommet may be provided with dampening properties by configuring at least a portion of it to be mechanically capable of absorbing, dampening, lessening, or preventing the transmission of vibrations from the ultrasound horn to the patient. Such mechanical configurations may comprise, but are not limited to, a dashpot, a coil spring, a leaf spring or any combination thereof. When a configuration with mechanical dampening properties is employed, the use of compounds with dampening properties may not be needed. The portion of the dampening grommet possessing dampening properties can be located between portions of the grommet not possessing dampening properties. Alternatively, the portion of the dampening grommet with dampening properties may be at the proximal end of grommet. Constructing the distal end of the dampening grommet as to possess dampening properties may also be effective. When the device is to be inserted through a lumen or open cavity of the body as to access an orifice leading into the treatment area, such as, but not limited to, when passing through the vagina to access the uterus, the dampening grommet should be constructed or configured as to protect the lumen or cavity through which it is passed from vibrations emitted from the ultrasound horn.

Depicted in FIG. 3 are exemplar configurations of dampening grommets for use with the ultrasonic liquid delivery device of the present invention. Though only a few specific exemplars are depicted, many configurations are possible. Consequently, the configurations depicted in FIG. 3 and described in detailed below are meant only to be illustrative and non-limiting. FIGS. 3A, B, C, and D depict exemplar configurations of a dampening grommet completely constructed from a compound possessing dampening properties. FIG. 3A depicts an external and cross-sectional view of a dampening grommet possessing a needle like configuration. Capable of fitting into small orifices, a needle like configuration is ideally suited for delivering liquids into treatment areas accessible through narrow orifices such as, but not limited to, the urethra, a soar or wound. The needle like grommet depicted in FIG. 3A, like all dampening grommets, contains channel 301 running from the proximal end to the distal end of the grommet. FIG. 3B depicts an external and cross-sectional view of a dampening grommet well suited for liquid delivery into treatment areas accessible by way of an intermediate or large orifice such as, but not limited to, the nostrils, anus, or vagina. FIG. 3C depicts an external and cross-sectional view of a dampening grommet with a double rounded distal end and a branching internal channel 301 that is well suited for delivering liquids into the sinuses through both of the patient's nostrils simultaneously. FIG. 2D depicts an external and cross-sectional view of a dampening grommet with a rounded configuration further comprising a second channel 302 well suited for douching a treatment area. Unlike channel 301, liquids from the liquid supply are not fed through channel 302. Rather, channel 302 serves as a point of evacuation for liquids delivered or fluids within the treatment area. Channel 302 may be connected to a collection reservoir or other means capable of collecting and containing spent liquids or discharged fluids.

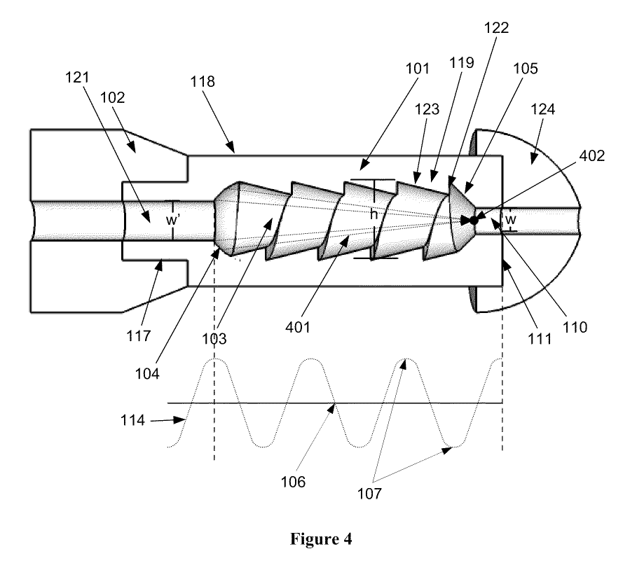

FIG. 4 illustrates a cross-sectional view of an alternative embodiment of ultrasound horn 101 further comprising slanted portion within front wall 105 of chamber 103 that serves to funnel the liquid to be expelled into channel 110. This results in a more efficient system of delivering liquids to the treatment area.

The embodiment depicted in FIG. 4 further comprising a concave portion within back wall 104 that forms an overall parabolic configuration in at least two dimensions. The ultrasonic vibrations depicted by arrows 401 emanating from concave portion back wall 104 travel in an undisturbed pattern of convergence towards the parabola's focus 402. As the ultrasonic vibrations 401 converge at focus 402, the liquid within chamber 103 is carried by vibrations 401 towards focus 402. The liquid passing through chamber 103 is therefore directed towards focus 402. Positioning focus 402 at or near the opening of channel 110, as to be in close proximity to the opening of channel 110 in front wall 105, consequently, may facilitate entry of the liquid into channel 110.

Positioning back wall 104 such that it lies approximately on an anti-node of the ultrasonic vibrations 114 passing through horn 101 may further facilitate entry of the liquid into channel 110 by the concave portions. Preferably, the center of back wall with concave portions lies approximately on an anti-node of the ultrasonic vibrations 114. It may also be desirable for the slanted portions of front wall 105 to form an angle equal to or greater than the angle of convergence of the ultrasonic vibrations emitted from the peripheral boundaries of the concave portions within back wall 104.

If the liquid passing through the embodiment of horn 101 depicted in FIG. 4 is to be atomized for inhalation or other delivery purposes, then the maximum width (w′) of channel 121 should be smaller than the maximum height (h) of chamber 103. Preferably, the maximum height of chamber 103 should be approximately twenty times larger than the maximum width of channel 121.

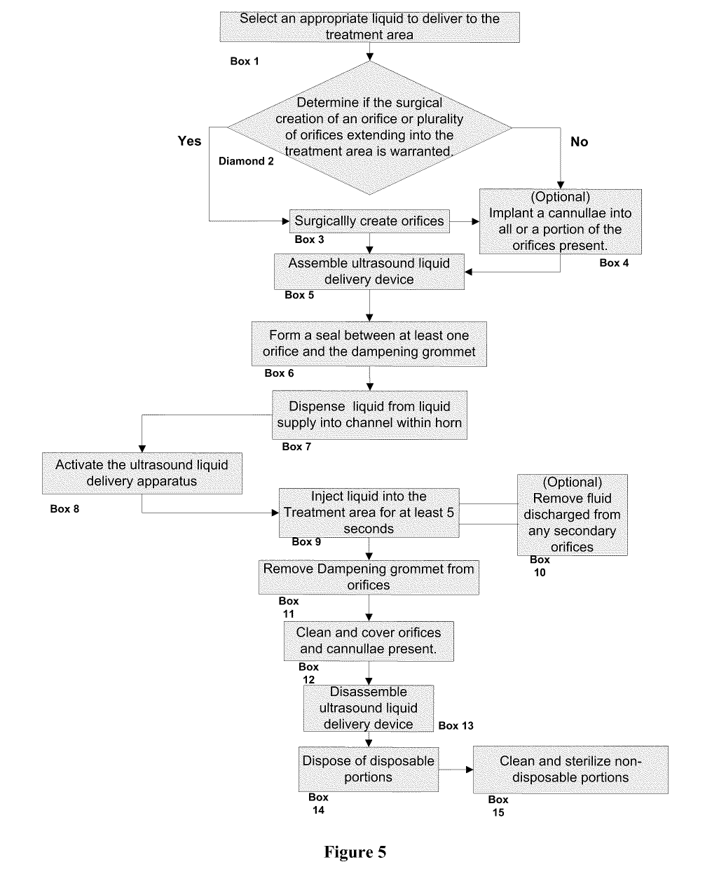

FIG. 5 contains a flow chart depicting a method of treating maladies within lumens, cavities or tissues of the body with the ultrasonic liquid delivery device of the present invention. In order to make the foregoing description more concrete in the reader's mind, the method is described with reference to treating a malady of bone marrow, such as, but not limited to, osteomyelitis and leukemia. It should be noted, that any tissue, cavity, or lumen of the body may be substituted for bone marrow in the foregoing description.

Preserving the integrity of bones, particularly bone marrow, is critically important for maintaining patient health, because bones are the site of red and white blood cell synthesis. Maladies within the bones, such as leukemia or osteomyelitis, can result in anemia and a compromised immune system. Treating such conditions often involves painful surgery due to the inability of drugs to adequately penetrate bone tissue. In the case of leukemia, the patient is often treated with painful bone marrow transplants during which unhealthy bone marrow is replaced with healthy bone marrow from a donor. With respect to the treatment of osteomyelitis, surgical debridement is the principal therapy. During the surgery, the bone is opened and the diseased tissue is scrapped away. The debridement procedure often leaves a large bony defect (dead space). Poorly vascularized, the presence of a dead space predisposes the patient to persistent infections. Consequently, dead bone tissue must be replaced with durable vascularized tissue. Debridement may need to be followed by stabilization such as external or internal fixation. Internal fixation devices often become infected resulting in painful complications to the patient. External fixation is labor intensive and requires an extended period of treatment averaging 8.5 months.

As indicated in Box 1, treating a malady of bone marrow with the present invention begins by first selecting the appropriate liquid to deliver to the bone marrow. Ideally the liquid chosen should provide a therapeutic benefit to the bone marrow such as, but not limited to, dissolving unwanted accretions, killing infectious organisms, arresting the growth of infectious organisms, killing cancerous cells, hindering the growth of cancerous cells, encouraging the growth of healthy cells, cleansing the bone marrow of infectious organisms, cleansing the bone marrow of diseased tissue, cleansing the bone marrow of necrotic tissue or any combination thereof. Ideally the liquid chosen should not harm or hinder the growth of healthy tissue any more than necessary to achieve the intended therapeutic benefit. A biopsy of the bone marrow may assist the user of the present of the invention in selecting the appropriate liquid. Culturing the removed bone marrow may allow the user to identify the infectious microbe causing the malady to be treated. A biopsy of the bone marrow may also allow the user of the present invention to identify the particular form of leukemia to be treated. Identifying the infectious microbe or particular leukemia present allows the user to select a liquid known or believed to be effective in treating the infection or leukemia present. Other manners of diagnosis or selection of an appropriate liquid are well known to those skilled in the healing arts and may be equally as effective.

In keeping with FIG. 5, after selecting an appropriate liquid, the user of the present invention, as depicted in Diamond 2, must then determine whether or not the creation of an orifice or plurality of orifices extending into the treatment area is warranted. If an orifice in the form of a sore, wound, or fissure extending into the bone marrow is already present, creation of additional orifices may not be necessary. However, if no such orifice is present, then an orifice extending into the bone marrow must be created, as depicted in Box 3. Creating multiple orifices extending into the bone marrow enables the user of the present invention to deliver the chosen liquid into the bone marrow from multiple sites. Additionally, the creation of multiple orifices enables the user of the present invention to flush or wash the bone marrow to be treated. Injecting a liquid into the bone marrow with the present invention forces the injected liquid through the bone marrow and out at least one secondary orifice present. Flushing or washing the bone marrow may be advantageous when the goal of the treatment is to remove necrotic tissue, cleanse the bone marrow of infectious organisms, cleanse the bone marrow of diseased tissue, removal undesirable elements from the bone marrow or any combination thereof. Orifices may be positioned at any position throughout the bone so long as they provide access to the bone marrow being treated.

As depicted in Box 4 of FIG. 5, orifices already present or created may be reinforced by implanting a cannula into the orifice. Implantation of a cannula into the orifice helps to prevent wound closure during the interval between successive treatments. Ideally, the implanted cannulae should extend into the bone marrow. Once all orifices have been created and cannulae inserted, if any, the present invention is completely assembled and prepared for operation, as depicted in Box 5, if not already done so. Assembling the present invention comprises the steps selecting and attaching an appropriate dampening grommet to the distal end of the ultrasound horn, filling a liquid supply with the chosen liquid, the connecting the liquid supply to a channel opening into the internal chamber of the horn, connecting said horn to an ultrasound transducer, connecting said transducer to a generator and connecting said generator to a power supply. Once the device has been assembled, orifices created, and cannulae, if any, implanted, a seal is then formed between at least one orifice and the dampening grommet, as depicted in Box 6. Forming the seal may be done by inserting the grommet into the orifice, or cannula if present, such that a seal forms between the outer sides of the grommet and the inner sides of the orifice or cannula. Alternatively, pressing the dampening grommet against a region of the patient's skin encompassing the orifice, or cannula if present, may also be done to form a seal between the distal end of the dampening grommet and the orifice or cannula. If a cannula has been implanted and the cannula extends out of the patient's body, forming a seal between the dampening grommet and an orifice may be accomplished by sliding the grommet down the cannulla such that a seal forms between the inner sides of the grommet and the outer sides of the cannula. The chosen liquid is then dispensed into a channel opening into the internal chamber, as depicted in Box 7. The ultrasound transducer is then activated, as depicted by Box 8. Following the activation of the ultrasound transducer the chosen liquid is then delivered to the bone marrow for at least approximately five seconds, as depicted by Box 9. Increasing the duration of liquid delivery, however, may be necessary depending on the severity of the malady being treated. If multiple orifices are present, the user of the present invention may wish to collect, wipe away, or otherwise remove any fluid discharged from any secondary orifices, as depicted in Box 10, simultaneously with the delivery of the liquid to the bone marrow. Collecting discharged fluid in a sealed reservoir may lessen the possibility of contaminating the surroundings of the treatment procedure.

In keeping with FIG. 5, following the completion of liquid delivery the ultrasound transducer is deactivated and the dampening grommet is removed from orifices against which it is sealed, as depicted in Box 11. The orifices or cannulae, if any, should then be cleaned and covered, as depicted in Box 12. Methods of cleaning and covering such orifices or cannulae are well known to those skilled in the healing arts. The ultrasound liquid delivery device of the present invention should then be disassembled, as depicted in Box 13. Disposable portions, which may comprise the dampening grommet, flexible hose, or liquid supply, should be disposed of, as depicted in Box 14. The remaining portions should be cleaned and sterilized, as depicted in Box 15. Methods of cleaning and sterilizing such elements are well known to those skilled in the healing arts. The procedure depicted in FIG. 5 should be repeated daily for approximately 10 days. However, depending on the severity of the malady to be treated, the frequency or number of treatment sessions may need to be increased, or could be decreased. After the final treatment session, any cannulae inserted should be removed.

It should be noted that the sequence of steps described above and depicted in FIG. 5 is merely a suggested sequence. Other combinations or sequences of the enumerated steps may be equally effective and are within the scope of the present invention. It should also be noted that the above procedure is applicable for treating lumens, cavities and tissues of the body other than bone marrow.

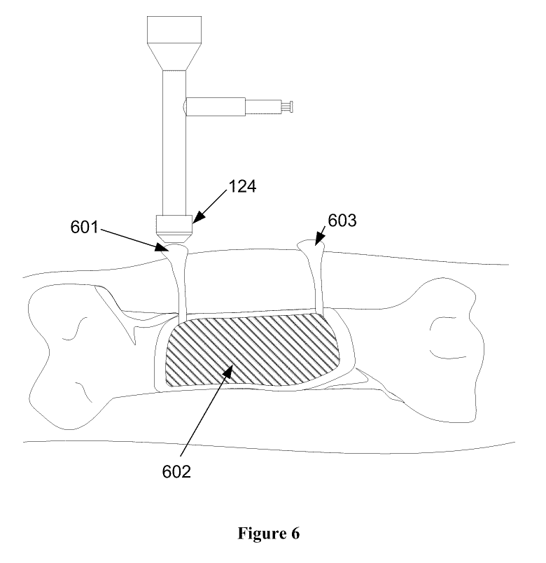

Depicted in FIG. 6 is an illustration of the treatment of bone marrow with the liquid delivery device of the present invention. Grommet 124 is inserted into a cannula 601, extending from the surface of the patient's skin to the bone marrow 602. Alternatively grommet 124 may be inserted into an orifice extending into the bone marrow 602. Cannula 603 is an optional additional cannula extending into the bone marrow which may serve as a point of egress for fluids within bone marrow or liquid delivered to the bone marrow. Cannula 603 may also serve as an additional orifice for liquid delivery.

It should be appreciated that elements described with singular articles such as “a”, “an”, and/or “the” and/or otherwise described singularly may be used in plurality. It should also be appreciated that elements described in plurality may be used singularly.

Although specific embodiments of apparatuses and methods have been illustrated and described herein, it will be appreciated by those of ordinary skill in the art that any arrangement, combination, and/or sequence that is calculated to achieve the same purpose may be substituted for the specific embodiments shown. It is to be understood that the above description is intended to be illustrative and not restrictive. Combinations of the above embodiments and other embodiments as well as combinations and sequences of the above methods and other methods of use will be apparent to individuals possessing skill in the art upon review of the present disclosure.

The scope of the claimed apparatus and methods should be determined with reference to the appended claims, along with the full scope of equivalents to which such claims are entitled.

I claim:

1. An ultrasound liquid delivery device comprising:
a. an ultrasound horn containing:
i. a proximal end opposite a distal end;
ii. an internal chamber containing a back wall, a front wall and side wall extending the back wall and front wall;
iii. a channel originating in the front wall of the chamber and ending in the distal end;
iv. a channel opening into the chamber at a location other than the font wall permitting a fluid to be delivered into the chamber; and
v. a protrusion extending from the side wall of the chamber containing a front facing edge and a rear facing edge more streamlined than the front facing edge; and
b. a dampening grommet attached to the distal end of the horn capable of blocking the delivery of ultrasonic vibrations from the distal end of the horn and containing a channel extending through the grommet in communication with the channel ending in the distal end.

2. The device according to claim 1 further characterized by the horn being capable of vibrating in resonance at a frequency of approximately 16 kHz or greater.

3. The device according to claim 2 further characterized by at least one point on the back wall of the internal chamber lying approximately on a node of the ultrasonic vibrations.

4. The device according to claim 2 further characterized by at least one point on a front-facing edge of the protrusion extending from the side wall of the chamber lying approximately on an antinode of the ultrasonic vibrations.

5. The device according to claim 2 further characterized by at least one point on the front wall of the internal chamber lying approximately on a node of the ultrasonic vibrations.

6. The device according to claim 1 further comprising a liquid supply coupled to the channel of the horn opening into the internal chamber.

7. The device according to claim 6 further comprising a flexible hose coupling the liquid supply to the channel of the horn opening into the internal chamber.

8. The device according to claim 1 further comprising a slanted portion within the front wall of the internal chamber.

9. The device according to claim 1 characterized by the protrusion extending from the side wall of the chamber being a discrete band encircling the chamber.

10. The device according to claim 1 characterized by the protrusion extending from the side wall of the chamber being a discrete band spiraling down the chamber.

11. The device according to claim 1 characterized by the channel originating in the front wall of the chamber having a maximum width smaller than the maximum height of the chamber.

12. The device according to claim 1 characterized by the maximum height of the internal chamber being approximately 200 times larger than the maximum width of the channel originating in the front wall of the internal chamber or greater.

13. The device according to claim 1 characterized by the channel opening into the chamber originating in the proximal end and opening into the back wall of the chamber and having a maximum width smaller than the maximum height of the chamber.

14. The device according to claim 13 further characterized by the maximum height of the internal chamber being approximately 20 times larger or greater than the maximum width of the channel opening into the back wall of the chamber.

15. The device according to claim 1 further comprising a concave portion within the back wall of the chamber.

16. The device according to claim 1 further comprising concave portions within the back wall of the chamber that form an overall parabolic configuration in at least two dimensions with the focus of the parabola lying in proximity to the opening of the channel originating within the front wall of the internal chamber.

17. The device according to claim 1 further comprising a transducer mechanically coupled to the proximal end.

18. The device according to claim 19 further comprising a generator driving the transducer.

19. The device according to claim 1 further characterized by at least a portion of the dampening grommet being formed from a compound possessing dampening properties.

20. The device according to claim 1 characterized by the dampening grommet occluding substantially all the surface of the distal end of the horn.
